(19) United States
(12) Patent Application Publication
(10) Pub. No.: US 2026/0283807 A1
(43) Pub. Date: Sep. 24, 2026

(54) KNEE ARTHROPLASTY IMPLANT COMPONENTS

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventor: Nathaniel Milton Lenz, Germantown, TN (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/474,234

(22) PCT Filed: May 23, 2024
(86) PCT No.: PCT/US2024/030711

§ 371 Date: Oct. 10, 2025

Related U.S. Application Data
(60) Provisional application No. 63/468,603, filed on May 24, 2023.
(60) Provisional application No. 63/540,596, filed on Sep. 26, 2023.

(51) Int. Cl. A61F 2/38; A61B 34/10; A61B 34/20; A61F 2/46

(52) U.S. Cl. CPC A61F 2/3859; A61B 34/10; A61B 34/20; A61F 2/389; A61F 2/4657; A61B 2034/105; A61B 2034/107; A61B 2034/108; A61B 2034/2055; A61F 2002/4633; A61F 2002/4668

(57) ABSTRACT

Disclosed herein are knee implant components (350) and a knee arthroplasty surgical method for implementing kinematic alignment of patient leg anatomy using asymmetric implant components and joint resections based on a leg mechanical axis (208). In one example, a surgical method may include aligning implant resections perpendicular to the leg mechanical axis to preserve constitutional varus or valgus while restoring the native tibial and femoral joint lines via use of asymmetric implant components. The constitutional varus or valgus may include an alignment of the knee to achieve pre-surgery tibial and/or femoral joint lines of the patient within an offset range. In some examples, the tibial resection is perpendicular to the leg mechanical axis and an asymmetric tibial implant is used and/or the femoral resection is perpendicular to the leg mechanical axis and an asymmetric femoral implant is used. Other examples are described.

### CROSS-REFERENCE TO RELATED APPLICATIONS

This application claims the benefit of U.S. Provisional Patent Application Ser. No. 63/468,603, filed May 24, 2023, and titled “KNEE ARTHROPLASTY MECHANICAL AXIS ALIGNMENT WITH REDUCED SHEAR LOADS,” and U.S. Provisional Patent Application Ser. No. 63/540,596, filed Sep. 26, 2023, and titled “KNEE ARTHROPLASTY IMPLANT COMPONENTS,” the entire contents of which applications are hereby incorporated by reference in their entireties.

### TECHNICAL FIELD

The present disclosure is directed to orthopedic implants and, more specifically, to a tibial component and/or femoral component of a knee arthroplasty implant system configured to implement post-surgical alignment of knee and other leg structures, for example, to provide a native alignment (e.g., varus or valgus) of the knee joint.

### BACKGROUND

Knee alignment is a foundation of successful knee arthroplasty procedures, for example, for a total knee arthroplasty (TKA). Proper alignment of the knee is considered one of the most influential factors in determining the long-term patient outcomes after TKA. A common approach is mechanical alignment, in which both femoral and tibial components are positioned perpendicular to the mechanical axis of each bone. Mechanical alignment (“MA”) is intended to obtain a hip-knee-ankle (HKA) angle of the limb of 180 degrees, which is generally considered neutral under standard weightbearing conditions. However, the HKA angle of the limb through MA often differs from the native knee, for example, as over 45 percent of a healthy population have been reported to have HKA more than 2 degrees from MA. Patients undergoing TKA using MA may require medial or lateral soft tissue adjustments.

Kinematic alignment has been used as an alternative approach to neutral MA. This approach aims to align the native (pre-arthritic) joint line along the three kinematic axes. Kinematic alignment is more patient-specific and, because this approach does not require changing the patient's anatomy, the integrity of the soft tissue envelope is preserved. Techniques for kinematic alignment generally involve resecting the distal end of the femur and/or the proximal end of the tibia to be non-perpendicular to the bone mechanical axis to achieve a varus or valgus alignment. For example, tibial joint line averages 3 degrees varus and the femoral joint line averages 3 degrees valgus relative to the mechanical axis. While TKA designs have built in a 3 degree joint line to the tibial and femoral implants based on these averages, a substantive portion of the population has varus/valgus joint line angles of greater than 3 degrees or unequal tibial and femoral joint line angles such that they result in varus or valgus HKA.

There remains a need for improved techniques for kinematic alignment of the knee joint during a knee arthroplasty procedure. It is with this in mind that the present disclosure is provided.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Disclosed herein are improved systems and methods for planning a knee arthroplasty procedure, including a knee arthroplasty surgical process for achieving or approximating kinematic alignment of patient leg anatomy.

In any preceding or subsequent example, a surgical method may include targeting a tibia resection perpendicular to a planned resultant leg mechanical axis, using an asymmetric thickness tibia implant to restore the tibia joint line relative to the tibia mechanical axis or leg mechanical axis within 1 or 2 degrees varus-valgus and restoring the femur joint line relative to the femur mechanical axis or leg mechanical axis within 1 or 2 degrees varus-valgus either through resections or through an asymmetric thickness femur implant with the bone resection perpendicular to the leg mechanical axis. In the present disclosure, resections to or other processes on the tibia refer to the proximal tibia (or proximal end of the tibia) arranged at the knee joint, and resections to or other processes on the femur refer to the distal femur (or distal end of the femur) arranged at the knee joint.

In general, a leg mechanical axis is a line from the center of the hip of a patient to the center of the ankle of the patient. A joint line generally represents a line or axis in the coronal plane describing the contacting regions of the articular surfaces. A femoral joint line may include a line between the lowest points of the medial and the lateral femoral condyles (including, for example, the lowest points of cartilage of the medial and the lateral femoral condyles). A tibial joint line may include a line corresponding with the medial and lateral sulci of the tibial plateau, (including, for instance, the thickness of cartilage of the medial and the lateral tibial plateau.

In any preceding or subsequent example, a knee arthroplasty surgical method may include kinematic alignment using asymmetric implant components and joint resections based on a leg mechanical axis.

In any preceding or subsequent example, the surgical method may include determining a target kinematic alignment for a patient. The target kinematic alignment may be or may be based on a constitutional, native, pre-arthritic, or presurgical alignment, including a varus or valgus alignment. In any preceding or subsequent example, the surgical method may include aligning implant resections perpendicular to the leg mechanical axis while preserving constitutional, native, pre-arthritic, or pre-surgical varus or valgus via use of asymmetric implant components.

In any preceding or subsequent example, the tibial resection is perpendicular to the leg mechanical axis. In any preceding or subsequent example, the femoral resection is perpendicular to the leg mechanical axis. In any preceding or subsequent example, the tibial resection and the femoral resection are perpendicular to the leg mechanical axis.

In any preceding or subsequent example, the tibial resection is perpendicular to the leg mechanical axis and the tibial implant component includes an asymmetric tibial implant. In any preceding or subsequent example, the femoral resection is perpendicular to the leg mechanical axis and the femoral implant component includes an asymmetric tibial implant. In any preceding or subsequent example, the tibial resection and the femoral resection are perpendicular to the leg mechanical axis, the tibial implant component includes an asymmetric tibial implant, and the femoral implant component includes an asymmetric femoral implant.

In any preceding or subsequent example, a method for planning a knee arthroplasty procedure for a patient may include obtaining patient-specific anatomical data of a limb, a prosthesis to be implanted in a knee of the limb, determining a leg mechanical axis of the limb, determining a pre-operative or target angle between a tibial mechanical axis and a tibial joint line, determining a resection plane of a tibia bone to implant the prosthesis, the resection plane is perpendicular to the leg mechanical axis of the limb, and a resulting angle between a resulting tibial mechanical axis and the tibial joint line is within a predetermined threshold of the pre-operative angle, and providing a surgical plan depicting the resection plane to be used. Resection planes perpendicular to the pre-arthritic leg mechanical axis will automatically restore the pre-arthritic HKA as long as the medial implant thickness (femur and tibia) is equal to the lateral implant thickness, which is common for TKA. Use of an asymmetric implant where the thinner lateral femur is offset by the thicker lateral tibia allows the femoral and tibial joint lines to also be restored within 1-2 degrees.

In any preceding or subsequent example, a knee arthroplasty method may include an alignment of the knee to achieve or approximate a pre-surgery tibial and femoral joint lines of the patient within an offset range.

In any preceding or subsequent example, the offset range is about 1 degree to about 2 degrees. In any preceding or subsequent example, the pre-surgery tibial and femoral joint lines are one of a varus angle or a valgus angle.

In any preceding or subsequent example, the proximal end of the tibia is resected perpendicular to the planned resultant leg mechanical axis and an asymmetric tibial implant is used to achieve or approximate the pre-surgery tibial joint line.

In any preceding or subsequent example, the distal end of the femur is resected perpendicular to the planned resultant leg mechanical axis and an asymmetric femoral implant is used to achieve or approximate the pre-surgery femoral joint line.

In any preceding or subsequent example, the proximal end of the tibia and the distal end of the femur are resected perpendicular to the planned resultant leg mechanical axis. In any preceding or subsequent example, an asymmetric femoral implant and an asymmetric tibial implant is used to achieve or approximate the pre-surgery tibial and femoral joint lines.

In any preceding or subsequent example, an asymmetric implant may be configured to be implanted on a prepared tibial surface that is perpendicular to the leg mechanical axis. In any preceding or subsequent example, an asymmetric implant may have a first side and a second side of different heights to achieve or approximate an angled joint line to implement a varus or valgus tibial and femoral joint line angles. In any preceding or subsequent example, an asymmetric tibial implant may have a tibial baseplate of varying thickness to achieve or approximate an angled joint line to implement a varus or valgus tibial joint line angle.

Surgical methods and asymmetric implant systems according to any preceding or subsequent example may provide multiple technological advantages. One non-limiting example of a technological advantage may include providing knee arthroplasty to a patient alignment (e.g., varus or valgus alignment) that maintains pre-surgical (or pre-arthritic) tibial and femoral joint lines within an offset with prepared tibial and/or femoral bone resections surfaces that are perpendicular to the leg mechanical axis. Conventional kinematic alignment techniques have involved the use of non-perpendicular tibial and/or femoral resections to achieve alignment. However, such techniques are prone to implant loosening, biomechanical issues and/or other system failures. Accordingly, technological advantages are achieved through the use of asymmetric implants according to some examples. In one non-limiting example of a technological advantage, surgical methods and asymmetric implant systems according to any preceding or subsequent example allow for alignment that reduces or even eliminates post-surgical biomechanical issues and/or implant loosening.

In any preceding or subsequent example, a total knee arthroplasty method may include determining a target joint angle for a knee joint of a patient; determining a mechanical axis associated with the knee joint; resecting at least one knee joint surface perpendicular to the mechanical axis, the at least one knee joint surface comprising at least one surface of one of a proximal tibia or a distal femur; and implanting at least one asymmetric implant on the at least one knee joint surface, the at least one asymmetric implant comprising a base portion configured to be arranged on the at least one knee joint surface and an asymmetric articulating surface configured to implement the target joint angle for the post-surgical knee joint.

In any preceding or subsequent example of the method, the target joint angle may be a pre-operative hip-knee-ankle (HKA) angle of the patient.

In any preceding or subsequent example of the method, the target joint angle may be one of a pre-operative varus or valgus knee joint angle.

In any preceding or subsequent example of the method, the target joint angle may be one of a pre-operative varus or valgus knee joint angle+/−an offset value.

In any preceding or subsequent example of the method, the mechanical axis may include a leg mechanical axis.

In any preceding or subsequent example of the method, the at least one asymmetric implant may be an asymmetric tibial implant, and the mechanical axis may be a leg mechanical axis.

In any preceding or subsequent example of the method, the at least one asymmetric implant may be an asymmetric femoral implant, and the mechanical axis may be a leg mechanical axis.

In any preceding or subsequent example of the method, at least one asymmetric implant may include an asymmetric tibial implant and an asymmetric femoral implant.

In any preceding or subsequent example, an asymmetric implant component may include a base portion configured to be arranged on a resected knee joint surface resected perpendicular to a mechanical axis associated with a knee joint of a patient; and an asymmetric articulating surface configured to engage a corresponding opposite implant component and to implement a target joint angle for the post-surgical knee joint.

In any preceding or subsequent example of the asymmetric implant, the target joint angle may be a pre-operative hip-knee-ankle (HKA) angle of the patient.

In any preceding or subsequent example of the asymmetric implant, the target joint angle may be one of a pre-operative varus or valgus knee joint angle.

In any preceding or subsequent example of the asymmetric implant, the target joint angle may be one of a pre-operative varus or valgus knee joint angle+/−an offset value.

In any preceding or subsequent example of the asymmetric implant, the mechanical axis may include a leg mechanical axis.

In any preceding or subsequent example of the asymmetric implant, the at least one asymmetric implant may be an asymmetric tibial implant, and the mechanical axis may be a leg mechanical axis.

In any preceding or subsequent example of the asymmetric implant, the at least one asymmetric implant may be an asymmetric femoral implant, and the mechanical axis may be a leg mechanical axis.

In any preceding or subsequent example of the asymmetric implant, the at least one asymmetric implant may include an asymmetric tibial implant and an asymmetric femoral implant.

In any preceding or subsequent example, a computer-assisted surgical system may include a display device; processing circuitry; and a memory coupled to the processing circuitry, the memory including instructions that, when executed by the processing circuitry, cause the processing circuitry to: receive a target joint angle for a knee joint of a patient, receive a mechanical axis associated with the knee joint, determine a resection surface perpendicular to the mechanical axis, the resection surface comprising at least one surface of one of a proximal tibia or a distal femur; determine an asymmetric implant for implanting on the resected surface, the asymmetric implant comprising a base portion configured to be arranged on the at least one knee joint surface and an asymmetric articulating surface configured to implement the target joint angle for the post-surgical knee joint, and generate a surgical plan for implanting the asymmetric implant on the patient to achieve or approximate the joint angle.

In any preceding or subsequent example of the computer-assisted surgical system, the target joint angle may be a pre-operative hip-knee-ankle (HKA) angle of the patient.

In any preceding or subsequent example of the computer-assisted surgical system, the target joint angle may be one of a pre-operative varus or valgus knee joint angle.

In any preceding or subsequent example of the computer-assisted surgical system, the mechanical axis may include a leg mechanical axis.

In any preceding or subsequent example of the computer-assisted surgical system, the at least one asymmetric implant may be an asymmetric tibial implant, and the mechanical axis may be a leg mechanical axis.

In any preceding or subsequent example of the computer-assisted surgical system, the at least one asymmetric implant may be an asymmetric femoral implant, and the mechanical axis may be a leg mechanical axis.

In any preceding or subsequent example of the computer-assisted surgical system, at least one asymmetric implant may include an asymmetric tibial implant and an asymmetric femoral implant.

Examples are not limited in this context. Additional technological advantages and technical features over conventional systems would be known to those of skill in the art based on the present disclosure.

Further features and advantages of at least some examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 depicts an operating theatre including an illustrative computer-assisted surgical system (CASS) in accordance with one or more features of the present disclosure.

FIGS. 2A-2C depict illustrative total knee arthroplasty (TKA) alignment configurations.

FIGS. 3A-3C depict surgical plans for treatment of a varus leg and a valgus leg using at least one asymmetric implants in accordance with one or more features of the present disclosure.

FIGS. 4A-4D depict surgical plans for treatment of a varus leg and a valgus leg using at least one asymmetric implants in accordance with one or more features of the present disclosure.

FIGS. 5A and 5B depicts an example of an asymmetric tibial implant in accordance with one or more features of the present disclosure.

FIG. 6 depicts simulation results of TKA implant components and TKA methods in accordance with one or more features of the present disclosure.

FIG. 7 illustrates an example of a surgical workflow in accordance with one or more features of the present disclosure.

It should be understood that the drawings are not necessarily to scale and that the disclosed examples are sometimes illustrated diagrammatically and in partial views. In certain instances, details which are not necessary for an understanding of the disclosed methods and devices, or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

Various features of an improved implant component, such as a tibial component and/or a femoral component, of a knee implant system and surgical methods for implanting the knee implant system will now be described more fully hereinafter with reference to the accompanying drawings, in which one or more features of the implant component will be shown and described. It should be appreciated that the various features may be used independently of, or in combination, with each other. It will be appreciated that an implant component as disclosed herein may be embodied in many different forms and should not be construed as being limited to the examples set forth herein. Rather, these examples are provided so that this disclosure will convey certain features of the implant component to those skilled in the art.

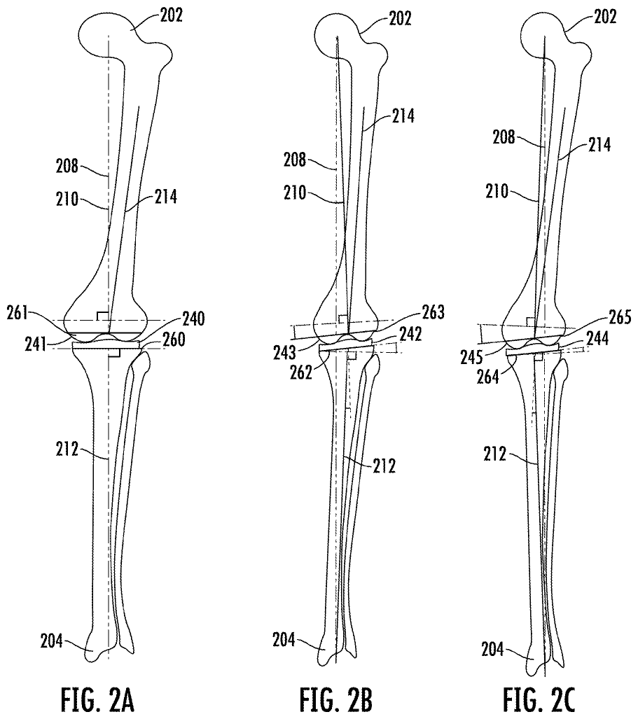

The present disclosure describes asymmetric implant components for a knee implant system. In general, an asymmetric implant component is configured to provide an angled joint line relative to the bone resections which may be aligned to a mechanical axis of a bone structure, such as a tibial mechanical axis (i.e., for a tibial component), a femoral mechanical axis (i.e., for a femoral component), and/or a leg mechanical axis. In general, a joint line represents a line or axis in the coronal plane describing the contacting regions of the articular surfaces (see, for example, FIGS. 2A-2C). A femoral joint line may include a line between the lowest points of the medial and the lateral femoral condyles (including, for example, the lowest points of cartilage of the medial and the lateral femoral condyles). A tibial joint line may include a line corresponding with the medial and lateral sulci of the tibial plateau, (including, for instance, the thickness of cartilage of the medial and the lateral tibial plateau).

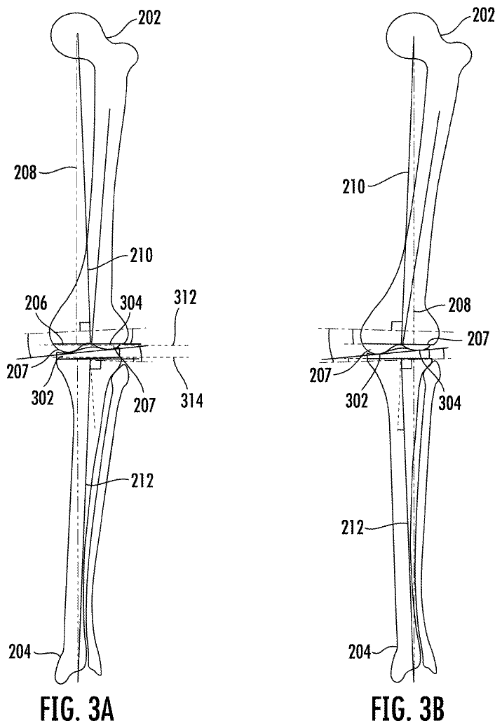

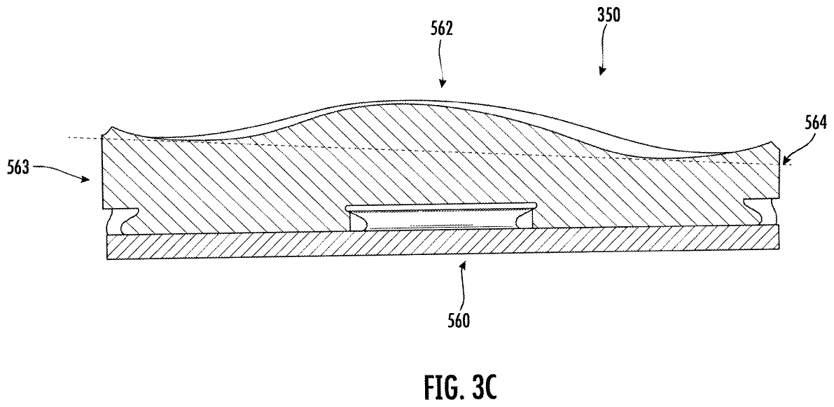

In some examples, a surgical process may use an asymmetric tibial component configured for a tibia resection perpendicular to the planned resultant leg mechanical axis and using an asymmetric thickness tibia implant to restore the tibia joint line relative to the tibia mechanical axis or leg mechanical axis (see, for instance, FIGS. 3A-3C). For example, the tibia joint line may be within a predetermined number of degrees varus-valgus (e.g., within about 1 to about 2 degrees or an “offset range”). In some examples, the offset or offset range may be determined based on a condyle offset, for instance, to achieve or approximate a pre-surgical condyle offset. The femur joint line may be restored relative to the femur mechanical axis or leg mechanical axis within a predetermined number of degrees varus-valgus either through resections or through an asymmetric thickness femur implant with the bone resection perpendicular to the leg mechanical axis. This surgical method may be enabled according to various examples through computer aided surgery, robotic surgery, patient specific guides, pre-operative planning with manual instrumentation, and/or the like.

The surgical processes and asymmetric implant components may provide multiple technological advantages over conventional techniques and devices. For instance, existing mechanical alignment approaches for TKA seek to resect the femur and tibia perpendicular to their respective mechanical axes which results in resections that are also perpendicular to the loading axis from the center of the hip to the center of the ankle. Placing resections perpendicular to the loading axis has the benefit of reducing shear forces at the implant-cement-bone or implant-bone interfaces, which is beneficial in reducing the risk of implant loosening. However, mechanical alignment can have negative impacts on knee biomechanics, because it does not entirely restore native lower limb alignment, alters the normal kinematics of knee motion, and typically requires medial or lateral soft tissue adjustments.

Alternative alignment techniques have been developed with the goal of leaving the leg in some amount of varus or valgus partially or fully matching the patient's pre-arthritic native alignment such that the native ligament lengths are more closely restored on both medial and lateral condyles. Some of these techniques also target restoring the pre-arthritic native joint line angle relative to the femur and tibia mechanical axes. When targeting the native leg alignment and joint line angles, conventional alternative alignment techniques, such as existing kinematic alignment processes, have little regard for resecting perpendicular to the loading axis of the leg, while some alternative alignments techniques depart from restoring the joint line on both femur and tibia to maintain leg alignment and a tibia resection perpendicular to the tibia mechanical axis. What these alternative alignments techniques do not address is when the leg is aligned in varus or valgus, the loading axis of the leg from the center of the hip to the center of the ankle rotates away from the mechanical axes of the femur and tibia.

In general, kinematic alignment is aimed at improved ligament balance and stability by preserving the native limb alignment and joint line and limiting ligament releases. However, with conventional techniques involving angled or inclined resection of the tibia and/or femur to achieve the desired joint angle, angled resection of the tibia (or femur) may increase the risk of loosening. For example, resection of more than 3 degrees from mechanical alignment with kinematic alignment may be particularly susceptible to implant component wear and loosening. Such concerns have led to restricted kinematic alignment which limits magnitude of the hip-knee-ankle (HKA) angle and the deviation of the tibia and femur resections from the tibia and femur mechanical axes in an effort to limit the shear loads at the tibia bone-to-implant interface.

Accordingly, because the bodyweight load vector or leg mechanical axis is a line from the hip to the ankle regardless of the hip-knee ankle angle, some examples may operate to align implant resections perpendicular to the leg mechanical axis when preserving constitutional varus or valgus, thereby causing less change in tibia medial-lateral shear compared to a 0 degree HKA than aligning the tibia perpendicular to the tibia mechanical axis.

Therefore, the features of the technical solutions described herein generally relate to optimizing implant alignment during surgical procedures for joint replacement, such as total knee arthroplasty (TKA). Some examples may include novel method of pre-operatively characterizing an individual patient's biomechanical function in order to optimize the alignment, positioning, and/or orientation of components of a knee prosthesis. Features of the technical solutions described herein are described in the context of TKA; however, the technical solutions described herein can be applicable to other types of surgical procedures, such as partial knee arthroplasty (PKA), and should not be limited only to a particular type of surgical procedure.

Generally, in total knee arthroplasty (TKA), poor post-operative patient outcomes are not caused by a poorly-designed prosthesis. Instead, the problem often stems from a well-designed prosthesis being installed in a less-than-optimal biomechanical position relative to the natural anatomy of the patient in an attempt to get the best anatomic fit. For instance, the probability of revision knee surgery due to pain or abnormal wear may be high even with a well-designed knee prosthesis if the prosthesis is misaligned or if the prosthesis is installed without considering the biomechanical effects of prosthetic orientation.

In some examples, implant components may be selected, designed, determined, and/or the like based, wholly or partially, on a computer-based surgical process. In various examples, surgical procedures for implanting a component may be determined, performed, and/or the like via the use of a computer-based surgical system. The alignment of a patient according to some examples may be determined via the use of a computer-based surgical system.

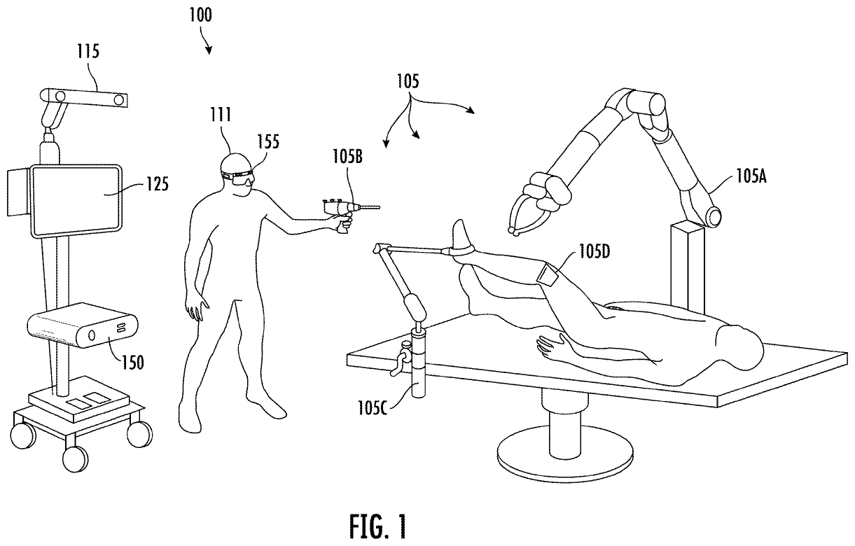

FIG. 1 provides an illustration of an example computer-assisted surgical system (CASS) 100, according to some examples. As described in further detail in the sections that follow, the CASS uses computers, robotics, and imaging technology to aid surgeons in performing orthopedic surgery procedures such as TKA or total hip arthroplasty (THA). For example, surgical navigation systems can aid surgeons in locating patient anatomical structures, guiding surgical instruments, and implanting medical devices with a high degree of accuracy. Surgical navigation systems such as the CASS 100 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An Effector Platform 105 positions surgical tools relative to a patient during surgery. The exact components of the Effector Platform 105 will vary, depending on the example employed. For example, for knee surgery, the Effector Platform 105 may include an End Effector 105B that holds surgical tools or instruments during their use. The End Effector 105B may be a handheld device or instrument used by the surgeon (e.g., a NAVIO® or CORI® handpiece or a cutting guide or jig) or, alternatively, the End Effector 105B can include a device or instrument held or positioned by a Robotic Arm 105A. While one Robotic Arm 105A is illustrated in FIG. 1, in some examples, there may be multiple devices. For example, there may be one Robotic Arm 105A on each side of an operating table T or two devices on one side of the table T. The Robotic Arm 105A may be mounted directly to table T, be located next to table T on a floor platform (not shown), mounted on a floor-to-ceiling pole, or mounted on a wall or ceiling of an operating room. The floor platform may be fixed or moveable. In one particular example, the robotic arm 105A is mounted on a floor-to-ceiling pole located between the patient's legs or feet. In some examples, the End Effector 105B may include a suture holder or a stapler to assist in closing wounds. Further, in the case of two robotic arms 105A, the surgical computer 150 can drive the robotic arms 105A to work together to suture the wound at closure. Alternatively, the surgical computer 150 can drive one or more robotic arms 105A to staple the wound at closure.

The Effector Platform 105 can include a Limb Positioner 105C for positioning the patient's limbs during surgery. One example of a Limb Positioner 105C is the SMITH AND NEPHEW SPIDER2 system. The Limb Positioner 105C may be operated manually by the surgeon or, alternatively, change limb positions based on instructions received from the Surgical computer 148 (described below). While one Limb Positioner 105C is illustrated in FIG. 1, in some examples, there may be multiple devices. For example, there may be one Limb Positioner 105C on each side of the operating table T or two devices on one side of the table T. The Limb Positioner 105C may be mounted directly to table T, be located next to table T on a floor platform (not shown), mounted on a pole, or mounted on a wall or ceiling of an operating room. In some examples, the Limb Positioner 105C can be used in non-conventional ways, such as a retractor or specific bone holder. The Limb Positioner 105C may include, for example, an ankle boot, a soft tissue clamp, a bone clamp, or a soft-tissue retractor spoon, such as a hooked, curved, or angled blade. In some examples, the Limb Positioner 105C may include a suture holder to assist in closing wounds.

The Effector Platform 105 may include tools, such as a screwdriver, light, or laser, to indicate an axis or plane, bubble level, pin driver, pin puller, plane checker, pointer, finger, or some combination thereof.

Resection Equipment 109 performs bone or tissue resection using, for example, mechanical, ultrasonic, or laser techniques. Examples of Resection Equipment 109 include drilling devices, burring devices, oscillatory sawing devices, vibratory impaction devices, reamers, ultrasonic bone cutting devices, radiofrequency ablation devices, reciprocating devices (such as a rasp or broach), and laser ablation systems. In some examples, the Resection Equipment 109 is held and operated by the surgeon during surgery. In other examples, the Effector Platform 105 may be used to hold the Resection Equipment 109 during use.

The Effector Platform 105 may also include a cutting guide or jig 105D that is used to guide saws or drills used to resect tissue during surgery. Such cutting guides 105D can be formed integrally as part of the Effector Platform 105 or Robotic Arm 105A, or cutting guides can be separate structures that can be matingly and/or removably attached to the Effector Platform 105 or Robotic Arm 105A. The Effector Platform 105 or Robotic Arm 105A can be controlled by the CASS 100 to position a cutting guide or jig 105D adjacent to the patient's anatomy in accordance with a pre-operatively or intraoperatively developed surgical plan such that the cutting guide or jig will produce a precise bone cut in accordance with the surgical plan.

The Tracking System 115 uses one or more sensors to collect real-time position data that locates the patient's anatomy and surgical instruments. For example, for TKA procedures, the Tracking System may provide a location and orientation of the End Effector 105B during the procedure. In addition to positional data, data from the Tracking System 115 can also be used to infer velocity/acceleration of anatomy/instrumentation, which can be used for tool control. In some examples, the Tracking System 115 may use a tracker array attached to the End Effector 105B to determine the location and orientation of the End Effector 105B. The position of the End Effector 105B may be inferred based on the position and orientation of the Tracking System 115 and a known relationship in three-dimensional space between the Tracking System 115 and the End Effector 105B. Various types of tracking systems may be used in various examples of the present disclosure, including, without limitation, Infrared (IR) tracking systems, electromagnetic (EM) tracking systems, video or image-based tracking systems, and ultrasound registration and tracking systems. Using the data provided by the tracking system 115, the surgical computer 150 can detect objects and prevent collision. For example, the surgical computer 150 can prevent the Robotic Arm 105A and/or the End Effector 105B from colliding with soft tissue.

Any suitable tracking system can be used for tracking surgical objects and patient anatomy in the surgical theatre. For example, a combination of IR and visible light cameras can be used in an array. Various illumination sources, such as an IR LED light source, can illuminate the scene allowing three-dimensional imaging to occur. In some examples, this can include stereoscopic, tri-scopic, quad-scopic, etc. imaging. In addition to the camera array, which in some examples is affixed to a cart, additional cameras can be placed throughout the surgical theatre. For example, handheld tools or headsets worn by operators/surgeons can include imaging capability that communicates images back to a central processor to correlate those images with images captured by the camera array. This can give a more robust image of the environment for modeling using multiple perspectives. Furthermore, some imaging devices may be of suitable resolution or have a suitable perspective on the scene to pick up information stored in quick response (QR) codes or barcodes. This can be helpful in identifying specific objects not manually registered with the system. In some examples, the camera may be mounted on the Robotic Arm 105A.

In some examples, specific objects can be manually registered by a surgeon with the system pre-operatively or intraoperatively. For example, by interacting with a user interface, a surgeon may identify the starting location for a tool or a bone structure. By tracking fiducial marks associated with that tool or bone structure or by using other conventional image-tracking modalities, a processor may track that tool or bone as it moves through the environment in a three-dimensional model.

In some examples, certain markers, such as fiducial marks that identify individuals, important tools, or bones in the theater, may include passive or active identifiers that can be picked up by a camera or camera array associated with the tracking system. For example, an IR LED can flash a pattern that conveys a unique identifier to the source of that pattern, providing a dynamic identification mark. Similarly, one or two-dimensional optical codes (barcode, QR code, etc.) can be affixed to objects in the theater to provide passive identification that can occur based on image analysis. If these codes are placed asymmetrically on an object, they can also be used to determine an orientation of an object by comparing the location of the identifier with the extent of an object in an image. For example, a QR code may be placed in a corner of a tool tray, allowing the orientation and identity of that tray to be tracked. Other tracking modalities are explained throughout. For example, augmented reality headsets can be worn by surgeons and other staff to provide additional camera angles and tracking capabilities.

In addition to optical tracking, certain features of objects can be tracked by registering physical properties of the object and associating them with objects that can be tracked, such as fiducial marks fixed to a tool or bone. For example, a surgeon may perform a manual registration process whereby a tracked tool and a tracked bone can be manipulated relative to one another. By impinging the tip of the tool against the surface of the bone, a three-dimensional surface can be mapped for that bone that is associated with a position and orientation relative to the frame of reference of that fiducial mark. By optically tracking the position and orientation (pose) of the fiducial mark associated with that bone, a model of that surface can be tracked with an environment through extrapolation.

The registration process that registers the CASS 100 to the relevant anatomy of the patient can also involve the use of anatomical landmarks, such as landmarks on a bone or cartilage. For example, the CASS 100 can include a 3D model of the relevant bone or joint, and the surgeon can intraoperatively collect data regarding the location of bony landmarks on the patient's actual bone using a probe that is connected to the CASS. Bony landmarks can include, for example, the medial malleolus and lateral malleolus, the ends of the distal femur and proximal tibia, and the center of the hip joint. The CASS 100 can compare and register the location data of bony landmarks collected by the surgeon with the probe with the location data of the same landmarks in the 3D model. Alternatively, the CASS 100 can construct a 3D model of the bone or joint without pre-operative image data by using location data of bony landmarks and the bone surface that are collected by the surgeon using a CASS probe or other means. The registration process can also include determining various axes of a joint. For example, for a TKA, the surgeon can use the CASS 100 to determine the anatomical and mechanical axes of the femur and tibia. The surgeon and the CASS 100 can identify the center of the hip joint by moving the patient's leg in a spiral direction (i.e., circumduction) so the CASS can determine where the center of the hip joint is located.

The CASS 100 may include a Tissue Navigation System 120 that provides the surgeon with intraoperative, real-time visualization of the patient's bone, cartilage, muscle, nervous, and/or vascular tissues surrounding the surgical area. Examples of systems that may be employed for tissue navigation include fluorescent imaging systems and ultrasound systems.

Although, as discussed herein, the majority of tracking and/or navigation techniques utilize image-based tracking systems (e.g., IR tracking systems, video or image-based tracking systems, etc.). However, electromagnetic (EM) based tracking systems are becoming more common for a variety of reasons. For example, implantation of standard optical trackers requires tissue resection (e.g., down to the cortex) as well as subsequent drilling and driving of cortical pins. Additionally, because optical trackers require a direct line of sight with a tracking system, the placement of such trackers may need to be far from the surgical site to ensure they do not restrict the movement of a surgeon or medical professional.

The Display 125 provides graphical user interfaces (GUIs) that display images collected by the Tissue Navigation System as well other information relevant to the surgery. For example, the Display 125 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The Display 125 may include, for example, one or more computer monitors. As an alternative or supplement to the Display 125, one or more members of the surgical staff may wear an Augmented Reality (AR) Head Mounted Device (HMD). For example, in FIG. 1 the Surgeon 111 is wearing an AR HMD 155 that may, for example, overlay pre-operative image data on the patient or provide surgical planning suggestions. Various example uses of the AR HMD 155 in surgical procedures are detailed in the sections that follow.

Surgical Computer 150 provides control instructions to various components of the CASS 100, collects data from those components, and provides general processing for various data needed during surgery. In some examples, the Surgical Computer 150 is a general-purpose computer. In other examples, the Surgical Computer 150 may be a parallel computing platform that uses multiple central processing units (CPUs) or graphics processing units (GPUs) to perform processing. In some examples, the Surgical Computer 150 is connected to a remote server over one or more computer networks (e.g., the Internet). The remote server can be used, for example, for storage of data or execution of computationally intensive processing tasks.

Various techniques generally known in the art can be used for connecting the Surgical Computer 150 to the other components of the CASS 100. Moreover, the computers can connect to the Surgical Computer 150 using a mix of technologies. For example, the End Effector 105B may connect to the Surgical Computer 150 over a wired (i.e., serial) connection. The Tracking System 115, Tissue Navigation System 120, and Display 125 can similarly be connected to the Surgical Computer 150 using wired connections. Alternatively, the Tracking System 115, Tissue Navigation System 120, and Display 125 may connect to the Surgical Computer 150 using wireless technologies such as, without limitation, Wi-Fi, Bluetooth, Near Field Communication (NFC), or ZigBee.

Pre-operatively, the CASS 100 can develop a proposed surgical plan based on a three-dimensional model of the joint and other information specific to the patient, such as the mechanical and anatomical axes of the leg bones, the epicondylar axis, the femoral neck axis, the dimensions (e.g., length) of the femur and hip, the midline axis of the hip joint, the ASIS axis of the hip joint, and the location of anatomical landmarks such as the lesser trochanter landmarks, the distal landmark, and the center of rotation of the hip joint. The CASS-developed surgical plan can provide a recommended optimal implant size and implant position and orientation based on the three-dimensional model of the joint and other information specific to the patient. The CASS-developed surgical plan can include proposed details on offset values, inclination and anteversion values, center of rotation, cup size, medialization values, superior-inferior fit values, femoral stem sizing and length.

FIGS. 2A-2C depict illustrative TKA alignment configurations. Depicted in FIGS. 2A-2C are a femur 202 and a tibia 204 associated with certain axes relating to knee alignment. The knee is the largest joint in the human body. The knee includes the distal or lower end of the femur 202 (thigh bone), the proximal or upper end of the tibia 204 (shin bone), and the inner surface of the patella (knee cap). The femur 202 rotates over the tibia 204, and the patella glides in a groove on the end of the femur 202 in front (i.e., anterior). The inner surface of these bony components is covered by cartilage (not shown) that, along with joint fluid, provides smooth motion of the joint and shock absorption. Various ligaments and muscles help in keeping the knee strong and stable.

Injuries, wear, and/or conditions such as arthritis cause the cartilage to wear away, leading to the knee becoming stiff and painful. In such conditions, bone can rub against bone, causing even more pain and loss of function. TKA, also known as knee replacement surgery, is the indicated treatment for many arthritic joint conditions and involves replacing the painful joint with an artificial prosthesis. During TKA, the proximal end of the tibia 204 and the distal end of the femur 202 are exposed by an incision made in front (i.e., anterior) of the knee. These bony structures are then cut and shaped (i.e., resected or prepared) to accept prosthetic implants.

During a TKA process, mechanical alignment seeks to resect the distal femur 202 and proximal tibia 204 perpendicular to their respective mechanical axes. The resections 206 may include a first resection of the femur 202 (femur resection) and a second resection of the tibia 204 (tibia resection).

FIG. 2A depicts a knee with neutral alignment (non-varus and non-valgus) with resections 260, 261 perpendicular to the leg mechanical axis 208. In the neutral aligned knee of FIG. 2A, the leg mechanical axis 208, the femoral mechanical axis 210, and the tibial mechanical axis 212 substantially coincide as shown As a result, FIG. 2A depicts a mechanical or natural alignment of the post-surgical knee in which a femur 202 is resected so that a resected surface of the femur 202 is substantially perpendicular (90 degrees) to a femoral mechanical axis 210, and the tibia 204 is resected so that a resected surface of the tibia 204 is substantially perpendicular (90 degrees) to a tibial mechanical axis 212. In the example of FIG. 2A, the tibial resection 260 and the femoral resection 261 are essentially perpendicular to the leg mechanical axis 208. The tibial implant 240 and the femoral implant 241 are essentially symmetric, leading to a mechanical alignment (“MA”) intended to obtain a hip-knee-ankle (HKA) angle of the limb of about 180 degrees, which is generally considered neutral under standard weightbearing conditions.

Loosening of the implant post-surgery is a challenge faced by patients undergoing such joint replacement surgeries. Features described herein address such challenges by reducing the occurrence of such implant loosening.

The leg mechanical axis 208 is a line from the center of the hip of the patient to the center of the ankle of the patient. The leg mechanical axis 208 of the human leg in the frontal plane is defined as a line drawn from the center of the femoral head to the center of the ankle joint. In a frontal plane, the leg mechanical axis 208 normally passes just medial to the center of the knee joint in the frontal plane. This line assumes sphericity in the femoral head and normal anatomy in the subtalar complex. In a sagittal plane, the normal mechanical axis runs from the center of the hip joint (or femoral head in the hip joint) to the center of the ankle joint. The leg mechanical axis 208, therefore, runs just behind the femoral head (because the femoral neck is generally anteverted about 15 degrees) and just in front of the knee.

Placing the resections perpendicular to the leg mechanical axis 208 reduces shear forces at the implant-cement-bone or implant-bone interfaces. Reducing such shear forces reduces the risk of implant loosening. Implant loosening is seen more commonly on the tibia 204 than on the femur 202. Hence, the orientation of the tibia resection relative to a tibial mechanical axis 212 is typically given higher priority than an alignment of the femur resection with a femoral mechanical axis 210 in some features.

Additionally, or alternatively, alignments may also be suggested with the goal of leaving the leg in some amount of varus or valgus partially or fully matching the patient's pre-arthritic (or pre-surgery) native alignment such that the native ligament lengths are more closely restored on both medial and lateral condyles. Knee joints usually present with different degrees of leg orientation because of various adaptations in cartilaginous and bony components of the joint. Compared to the idealized knee anatomy, there are typically two different types of alignment: (1) varus alignment (i.e., bow-legged) and (2) valgus alignment (i.e., knock knees). A goal of the TKA procedure is to restore optimal biomechanical alignment in a reconstructed joint. This is because a properly aligned joint will have better function, result in higher patient satisfaction, and increase the longevity of the reconstructed joint.

Kinematic alignment seeks to restore the pre-surgery native joint line angle relative to the femoral mechanical axis 210 and tibial mechanical axis 212. As shown in FIGS. 2B and 2C, when targeting the native leg alignment and joint line angles with symmetric implants, conventional techniques for the resections being of the tibia 263, 265 and/or the femur 262, 264 are not perpendicular to the leg mechanical axis 208, so surgeons either fail to minimize shear forces at the bone to implant interfaces by aligning the implants relative to the joint line, for example, departing from restoring the joint line on both the femur 202 and the tibia 204 to maintain leg alignment and a tibia resection perpendicular to the tibial mechanical axis 212, or they choose a compromise and align the tibia cut perpendicular to the tibia mechanical axis and deviate from the femur mechanical axis to achieve some amount of varus or valgus HKA. However, a technical challenge with such alignments is that when the leg is aligned in varus or valgus, the leg mechanical axis 208 from the center of the hip to the center of the ankle rotates away from the mechanical axes (210, 212) of the femur 202 and tibia 204. Solutions provided herein address such technical challenges.

As used herein, a vertical line that extends distally from the center of the pubic symphysis is known as a “vertical axis.” The leg mechanical axis 208 of the leg (or limb) is determined by drawing a line from the center of the femoral head to the center of the ankle joint. The femoral mechanical axis 210 runs from the head of the femur 202 to the intercondylar notch of the distal femur, and the tibial mechanical axis 212 extends from the center of the proximal tibia to the center of the ankle. In some cases, the leg mechanical axis 208 coincides with the femoral mechanical axis 210 and the tibial mechanical axis 212. The medial angle formed between the femoral mechanical axis 210 and the tibial mechanical axis 212 is called the hip-knee-ankle (HKA) angle, which represents the overall alignment of the lower extremity and is usually slightly less than 180 degrees in normal knees.

FIGS. 3A and 3B respectively depict surgical plans for treatment of a varus leg and a valgus leg using at least one asymmetric implants according to some examples, where the tibia and femur resections are perpendicular to the leg mechanical axis and the tibia and femur joint lines are restored using asymmetric thickness tibia and femur. The asymmetric thickness of the resections 206 is shown by the shaded portions 207. The resections 206 are “asymmetric,” wherein a first portion of the resection of the bone (tibia 204) that is on a first side (e.g., lateral side) of the bone's mechanical axis (e.g., tibial mechanical axis 212) is larger than a second portion of the resection that is on the other side (e.g., medial side) of the bone's mechanical axis. For the femur, for femoral “asymmetric” resections, a first portion of the resection of the bone (femur 202) that is on a first side (e.g., medial side) of the bone's mechanical axis (e.g., femoral mechanical axis 210) is larger than a second portion of the resection that is on the other side (e.g., lateral side) of the bone's mechanical axis.

In FIG. 3A and FIG. 3B, the asymmetric resections 206 of the femur 202 are depicted using shaded portions. The femoral mechanical axis 210 divides the resections 206 such that a first resection portion 302 is on the left of the femoral mechanical axis 210, and a second resection portion 304 is on the right of the femoral mechanical axis 210. As can be seen, the first resection portion 302 and the second resection portion 304 are of different dimensions, e.g., height from the knee joint. Other dimensions of portions 302 and 304 also vary, such as area, volume, etc. The femur 202 is resected so that the surface of the femur 202 after resecting is substantially perpendicular to the leg mechanical axis 208.

The alignment that facilitates minimized tibia interface shear with improved ligament balance and stability by preserving the native limb alignment and joint line and limiting ligament releases is achieved by performing resections 206 substantially perpendicular to the leg mechanical axis 208 with an asymmetric implant as described. However, preserving the native HKA with this alignment may increase the loading on one side of the tibia (medial or lateral) compared to the other and therefore increase the risk of loosening. To address such concerns, a restricted alignment is performed in one or more features, in which the magnitude of the hip-knee-ankle (HKA) angle is limited to restrict the deviation of the leg mechanical axis (208) from the tibial mechanical axis 212 and the femoral mechanical axis 210, respectively. Such restrictions limit the asymmetric loads at the tibia bone-to-implant interface. Because the bodyweight load vector or leg mechanical axis 208 is a line from the hip to the ankle regardless of the hip-knee ankle angle, aligning the implant resections 206 perpendicular to the leg mechanical axis 208 when preserving constitutional varus or valgus causes less change in tibia medial-lateral shear compared to a 0 degrees HKA than aligning the tibia 204 perpendicular to the tibial mechanical axis 212.

FIG. 3D depicts an asymmetric tibial implant according to various examples, more specifically, a posterior section through an asymmetric tibial implant. As shown in FIG. 3D, an asymmetric tibial implant 350 may include a base or tray 560 and an asymmetric surface 562 (e.g., a spacer, articulating surface, etc.) configured to engage the proximal end of the femur or a corresponding femoral component. As shown in FIG. 3D, asymmetric surface 562 has a first side 563 and a second side 564 of different heights. For example, the height of the first side is greater than the height of the second side 564 to cause the asymmetric surface 562 to be asymmetric. The heights of the first side 563 and the second side 564, or the height difference thereof, may be determined to achieve or approximate a certain HKA or joint line angle (or varus or valgus angle). In various examples, the higher side may be placed laterally or medially depending on the type of angle being implemented for the patient (i.e., a varus or valgus angle).

In various examples, a femoral component may be similarly configured with sides or other components of different heights. For example, the height of and/or the height difference between the medial condyle portion and the lateral condyle portion of a femoral component may be determined to achieve or approximate a certain HKA or joint line angle (or varus or valgus angle).

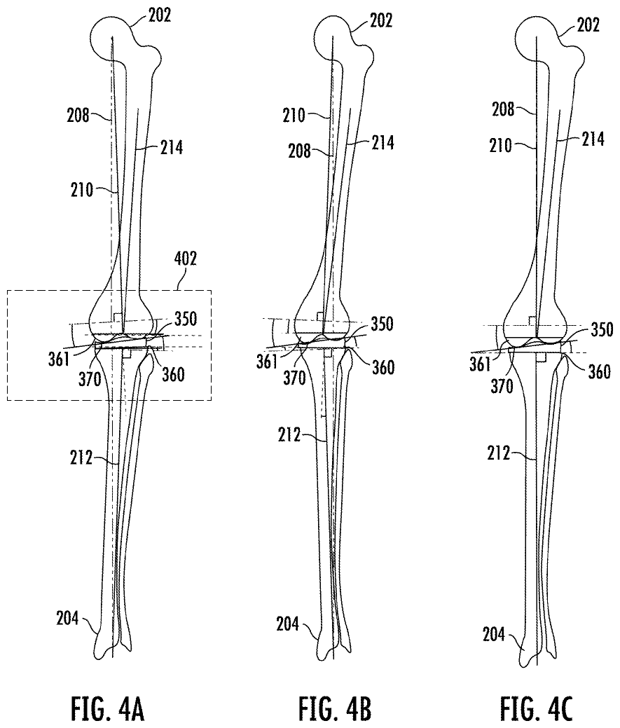

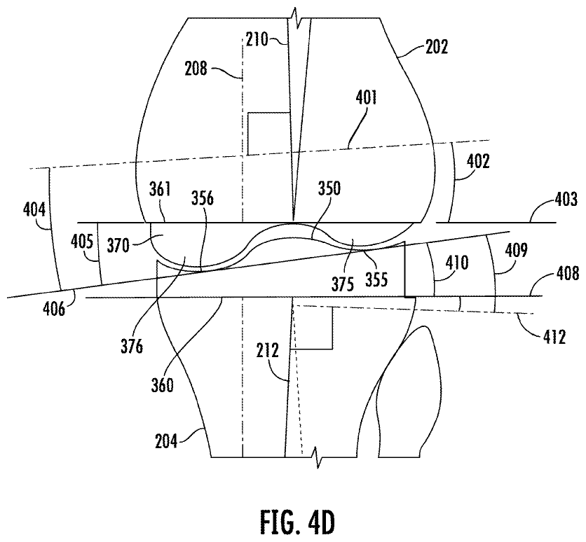

FIGS. 4A-4C depict illustrative asymmetric implant devices implanted to achieve or approximate kinematic alignment according to some examples. As shown in FIG. 4A, surgical processes according to some examples are implemented to achieve a target kinematic alignment for a patient. The target kinematic alignment may be varus or valgus. In some examples, the target kinematic alignment may be determined based on the pre-surgical alignment of the patient+/−an offset value. In other examples, the target alignment may be determined based on various optimization techniques, for instance, based on kinematic simulation of the post-surgical knee of the patient.

For example, a patient may have a pre-surgical alignment of 15 degrees. The surgical process may attempt to achieve a similar alignment, however, optimized based on various factors. For instance, it may be determined that 13 degrees is the optimal alignment for the patient based on kinematic simulation of the post-surgical knee of the patient. In some examples, the target alignment may be determined via a CASS or through other automated and/or manual processes.

In FIG. 4A, a varus knee is depicted with tibia resection 360 perpendicular to the leg mechanical axis 208 and asymmetric tibial implant 350, and femoral resection 361 perpendicular to the leg mechanical axis 208 and asymmetric femoral implant 370. FIG. 4B shows a valgus knee with tibia resection 360 perpendicular to the leg mechanical axis 208 and asymmetric tibial implant 350, and femoral resection 361 perpendicular to the leg mechanical axis 208 and asymmetric femoral implant 370. FIG. 4C shows a neutral knee with tibia resection 360 perpendicular to the leg mechanical axis 208 and asymmetric tibial implant 350, and femoral resection 361 perpendicular to the leg mechanical axis 208 and asymmetric femoral implant 370

Although FIGS. 4A-4C depict both the tibial resection 360 and the femoral resection 361 as being perpendicular to the leg mechanical axis 208, examples are not so limited as only one of the tibial resection 360 or the femoral resection 361 may be perpendicular to the leg mechanical axis 208. For example, in the examples of any of FIGS. 4A-4C, only the tibial resection 360 may be perpendicular to the leg mechanical axis 208 and only the tibial implant 350 may be asymmetric (for example, the femur may be resection non-perpendicular to the leg mechanical axis 208 and the femoral component may be a symmetric or otherwise conventional femoral implant component).

In some examples, the surgical computer 148 provides a surgical plan that suggests and facilitates resecting the femur 202 and/or the tibia 204 in relation to the leg mechanical axis 208 (instead of the femoral mechanical axis 210 and/or the tibial mechanical axis 212). In some examples, the targeting of the tibial resections 360 perpendicular to the leg mechanical axis 208 includes using an asymmetric thickness tibial implant 350 to restore the tibia joint line relative to the tibial mechanical axis 212 or the leg mechanical axis 208 within a predetermined target angle (e.g., 1 or 2 degrees varus-valgus). In some examples, targeting the femoral resection 361 further includes restoring the femur joint line relative to the femoral mechanical axis 210 or the leg mechanical axis 208 within a predetermined target angle (e.g., 1 or 2 degrees varus-valgus) either through an asymmetric thickness femur implant 370 with the bone resection perpendicular to the leg mechanical axis 208. In some examples, the surgical method provided by the surgical plan is enabled through computer-aided surgery, robotic surgery, patient-specific guides, and/or pre-operative planning with manual instrumentation.

FIG. 4D depicts a detailed view of area 402 of FIG. 4A. As shown in FIG. 4D, femoral implant 370 includes a lateral condyle 375 and a medial condyle 376. The tibial component 350 includes a lateral portion 355 configured to receive or otherwise engage with the lateral condyle 375 during movement of the knee joint, and a medial portion 356 configured to receive or otherwise engage with the lateral condyle 376 during movement of the knee joint.

The asymmetric property of the implant devices may be based on or measured with respect to various factors. In some examples, with respect to the tibial implant 350, an articulation line 406 running through the lateral portion 355 and the medial portion 356 (for instance, the lowest point or surface of the lateral portion 355 and the medial portion 356) may form an angle 410 with respect to the resection 360 (or a line 408 extending parallel to the resection 360) or an angle 409 with respect to a line 412 extending perpendicular to the tibial mechanical axis 212. In some examples, angle 409 or angle 410 may be the target angle or may be associated with the target angle (e.g., a varus/valgus target angle).

In some examples, with respect to the femoral implant 370, the articulation line 406 may run through the lateral condyle 375 and the medial condyle 376) and may form an angle 405 with respect to the resection 361 (or a line 403 extending parallel to the resection 361) or an angle 404 with respect to a line 401 extending perpendicular to the femoral mechanical axis 210. In another example, an angle 402 may be formed between the resection 361 and the line 401 extending perpendicular to the femoral mechanical axis 210. In some examples, angle 402, angle 404, or angle 405 may be the target angle or may be associated with the target angle (e.g., a varus/valgus target angle).

The surgical plans for femoral coronal alignment (for instance, varus-valgus) may be configured to achieve one or more procedural features or optimizations. Non-limiting examples of femoral coronal alignment features may include a lateralized anterior trochlea, restoring pre-arthritic or pre-surgical distal condyle offset (for instance, most often larger medial than lateral offset which lateralizes distal trochlea, which can also affect high flexion medial-lateral laxity balance opposite to low flexion balance), restoring native trochlea sulcus position and orientation, medial-lateral placement goals (for instance, avoiding mediolateral overhang or medialization of the femoral component (increased Q-angle), lateral undercoverage (anterior cut), and/or centralized patella contact in extension and patellofemoral contact on medial and lateral facets in flexion.

The surgical plans for tibial coronal alignment (for instance, varus-valgus) may be configured to achieve one or more procedural features or optimizations. Non-limiting examples of tibial coronal alignment features may include a resection or cut perpendicular to load axis (for example, hip center-to-center of ankle), restoring pre-surgical or pre-arthritic proximal tibia condyle offset (most often smaller medial than lateral offset, using a tibia stem that fits within bone, and medial lateral placement.

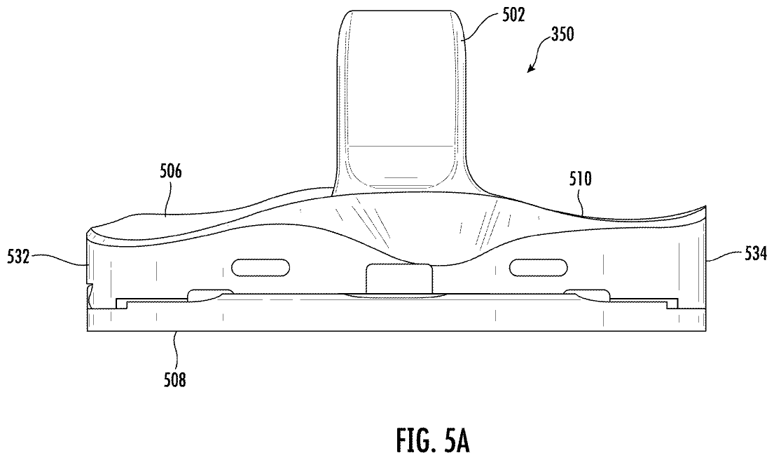

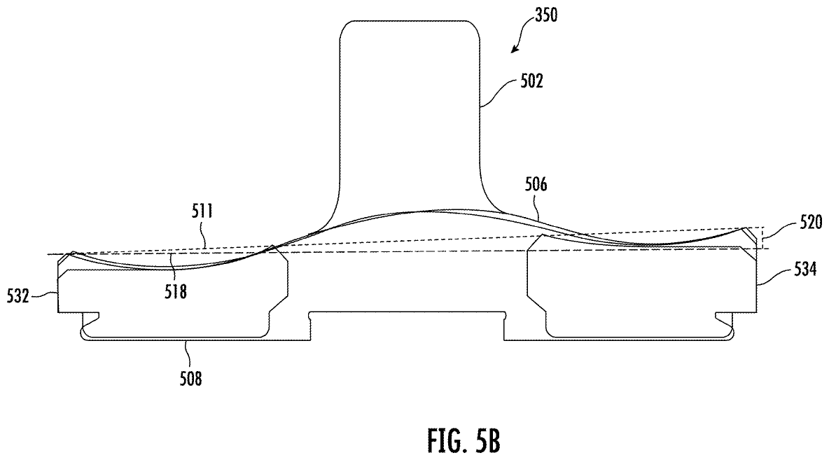

FIGS. 5A and 5B depicts an example of an asymmetric tibial implant in accordance with one or more features of the present disclosure. As shown in FIG. 5A, an asymmetric tibial implant 350 may include a bottom plate or base 508 configured to be implanted on a resected surface of a proximal end of a tibia. A tibial tray 510 may be coupled to the plate 508 having a first side 532 (for instance, a lateral side) and a second side 534 (for instance, a medial side). The plate 508 may be arranged and configured to engage with a corresponding femoral component of a knee implant system, for example, via a tibial post 502 and an articulation surface 506.

Referring to FIG. 5B, therein is depicted cross-sections of tibial implants for cruciate retaining (blue), posterior stabilized (orange), or bicruciate retaining (green) configured similar to 350 of FIG. 5A. The articulation surface 506, or portions thereof configured to engage a corresponding femoral component, may be asymmetric or angled to achieve or approximate a varus or valgus HKA angle when the tibial implant 350 is installed on a resected proximal tibial surface perpendicular to an axis, such as a leg mechanical axis, tibial mechanical axis, or a femoral mechanical axis. Line 518 is perpendicular to the tibial resected surface (not shown) and line 511 intersects the different surface heights of the articulation surface, creating an angle 520 between line 518 and line 511. The asymmetry of tibial tray 508 can be based on other features of articulation surface, such as the lowest points of the articulation surface 506, differences between medial and lateral condyle contact points, and/or the like.

In some examples, the asymmetry of articulation surface may be configured to achieve an HKA angle when the tibial implant 350 is installed on a resected proximal end of the tibia, including a resected surface that is perpendicular to the leg mechanical axis or the tibia mechanical axis. In various examples, as long as the combined thickness of the femur implant and tibial implant of the medial side matches the combined thickness of the lateral side and the cuts are perpendicular to the leg mechanical axis, the HKA will be restored or approximately restored to the native HKA. For instance, asymmetric thickness implants with varying asymmetry may be used to match or approximately match the native femur joint line angle and/or the native tibia joint line angle.

### Case Study: Simulation Analysis

A computational analysis study was performed to generate simulation results for surgical techniques according to some examples. The computational analysis was performed simulating 0-120 degree knee flexion of a PS TKA with a 3 degree asymmetric joint line using previously validated software (LIFEMOD® simulation software from Smith & Nephew, Inc) that includes MCL, LCL, popliteal-fibular ligament, iliotibial band, iliopatellar band, and medial retinaculum.

Scenarios for several predetermined deviations, such as 0 degree, 3 degree, 5 degree, 7 degree, 8 degree, 10 degree, etc., constitutional varus and valgus were created by moving the hip and ankle joints medial for varus and lateral for valgus from their mechanical alignment positions. The quadriceps origin was moved with the hip joint, while all anatomy at the knee joint was unchanged for comparison purposes. Each constitutional alignment was evaluated with tibia 204 perpendicular to the tibial mechanical axis 212 and to the leg mechanical axis 208 (FIG. 3A). The femur 202 was aligned relative to the tibia 204 for rectangular flexion-extension gaps. The tibia bone-to-implant medial-lateral shear load was compared for each analysis. The resulting tibia joint line orientation was calculated.

Tibia bone-to-implant medial-lateral shear load increased in the medial direction with knee flexion for all scenarios and implant alignments. Tibia shear was more medial for constitutional varus and less medial for constitutional valgus when the tibia implant was aligned perpendicular to the tibia bone mechanical axis than when aligned to the leg mechanical axis (FIG. 2B). Tibia medial-lateral shear was similar for the different deviations, such as the 0 degree, 5 degree, and 10 degree varus, but increased with increasing valgus when the implant was aligned to the leg mechanical axis 208. The femur and tibia joint line angles for the one or more deviations (e.g., 5 degree, 7 degree, etc.) varus and valgus leg mechanical alignment scenarios are similar to the average constitutional varus and valgus population.

The following Table 1 provides femur and tibia joint line angles for 10 degrees varus to 10 degrees valgus HKA scenarios when aligning the joint resections to the tibia mechanical axis or the leg mechanical axis for a 3 degrees asymmetric joint line TKA.

TABLE 1

| | | Tibia Mechanical Aligned | | Leg Mechanical Aligned | |
|---|---|---|---|---|---|
| | | Mechanical | | Mechanical | |
| | | Lateral Distal | Medial Proximal | Lateral Distal | Medial Proximal |
| Scenario | | Femoral Angle (°) | Tibia Angle (°) | Femoral Angle (°) | Tibia Angle (°) |
| 10° | varus | 97 | 87 | 92 | 82 |
| 5° | varus | 92 | 87 | 89.5 | 84.5 |
| 0° | | 87 | 87 | 87 | 87 |
| 5° | valgus | 82 | 87 | 84.5 | 89.5 |
| 10° | valgus | 77 | 87 | 82 | 92 |

| | | |
|---|---|---|
| Avg. varus 4.45° [1] | 89.03 | 85.13 |
| Avg. valgus 3.56° [1] | 85.45 | 89.06 |

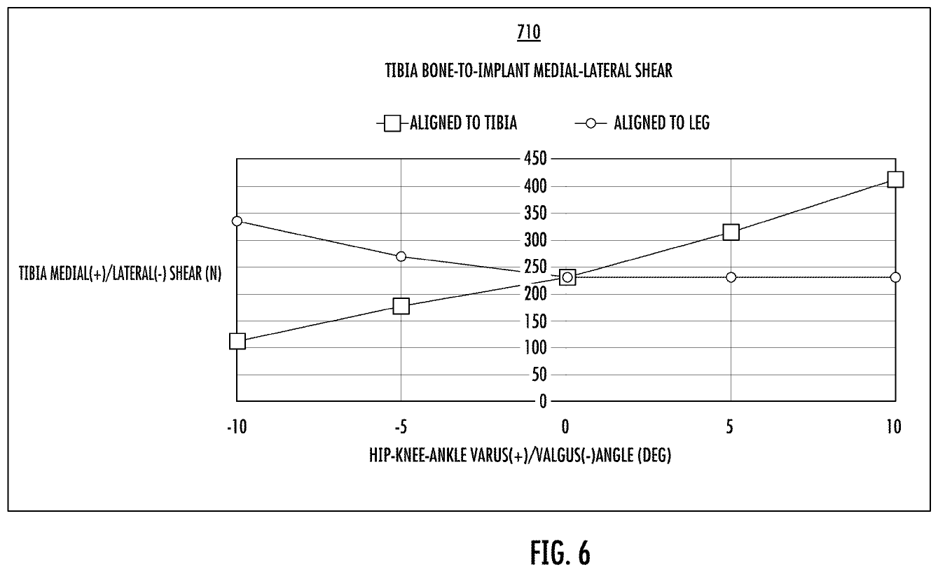

FIG. 6 depicts the results of tibia shear versus HKA angle when aligning tibia resection to the tibial mechanical axis 212 or the leg mechanical axis 208. Further, Table 1 provides example results for comparison of femur and tibia joint line angles for 10 degrees varus to 10 degrees valgus HKA scenarios when aligning the joint resections to the tibia mechanical axis or the leg mechanical axis for a 3 degree asymmetric joint line TKA. The depicted results in FIG. 6, Table 1, and elsewhere herein are examples and other resulting values are possible depending on specific experimental values.

When aligning a TKA in constitutional varus, the tibia bone-to-implant medial-lateral shear load is reduced by aligning the tibia implant perpendicular to the leg mechanical axis 208 running between the hip and ankle centers, which is varus to the tibial mechanical axis 212. Furthermore, the resulting joint line when using an asymmetric joint line implant is close to average anatomical data when the tibia 204 is aligned to the leg mechanical axis 208. Hence, as per the features of the technical solutions described herein, the kinematic alignment of an asymmetric joint line implant results in less risk of tibia loosening.

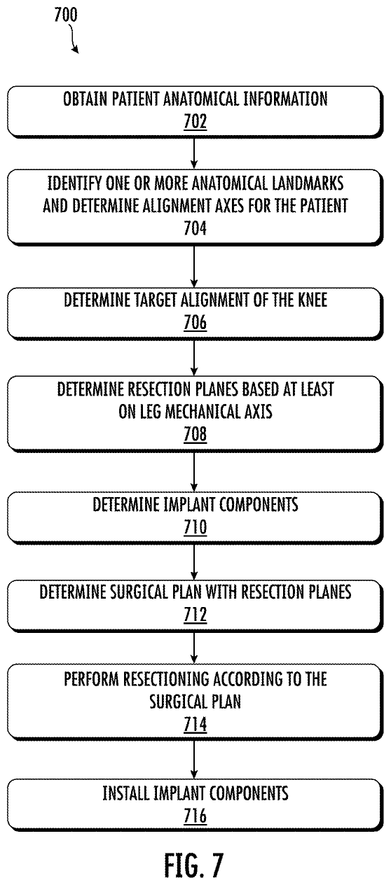

FIG. 7 depicts a flowchart of a method of providing a patient-specific surgical plan for a joint replacement procedure, such as a TKA procedure, according to one or more features described herein.

In particular examples, the method includes obtaining patient-specific anatomical data of a patient's joint at block 702. For example, a full scan of the patient's limb is captured. For economical or efficiency purposes, however, one or more partial scans of a patient's limb may be used rather than a full limb scan. In this regard, the exact location of each partial scan relative to a patient's limb may be carefully noted to ensure that the scans are spaced apart in all directions correctly before determining one or more of the indicators and/or axes (e.g., the leg mechanical axis 208) provided herein. Furthermore, one or more partial scans may be utilized when a particular landmark, such as the true femoral head center or the transepicondylar axis, cannot be determined and/or has been compromised, for example, due to injury, deformation, or other reasons.

The patient-specific anatomical data of the knee is obtained when the limb is in full extension (i.e., approximately 180 degrees). However, it would be appreciated that this may not be possible or feasible in all patients, owing, for example, to the presence of pre-existing disease, injury, or deformities of the limb. By way of example, a patient with a flexion deformity or contracture of the knee may be physically unable to fully straighten or extend the knee. Accordingly, in certain examples, the anatomical data of the limb is obtained when the limb is not fully extended.

In particular examples, the method of the present feature further includes the initial step of determining information, such as angles, axes, etc., from a patient-specific image of the knee. Suitably, a patient-specific image, and hence patient-specific anatomical data, is obtained pre-operatively and/or intra-operatively using one or more non-invasive imaging modalities, such as radiological imaging, computerized tomography (CT)/computerized axial tomography (CAT), magnetic resonance imaging (MRI), inclusive of full limb MRI, ultrasound and/or other conventional means. The patient-specific anatomical data obtained therefrom may then be pre-processed and/or converted to form a patient-specific model or image of the knee joint. Such a patient-specific model may include a two-dimensional model (e.g., radiographs, 2D slices of MRI) and/or a three-dimensional model (e.g., a three-dimensional (3D) computer-aided design (CAD) model). Preferably, the patient-specific anatomical data is determined or measured from a radiograph or radiological image. Even more preferably, the patient-specific image is or comprises a long-leg radiograph.

At block 704, one or more anatomical landmarks are identified, and alignment axes for the patient are determined based on the anatomical data. With regards to the femoral and/or tibial shafts, any central portion(s) or point(s) along the shaft, such as proximal, middle, or distal central portions, may be used as the anatomical indicators in performing the method of the present feature. Suitably, the patient-specific image and the patient-specific anatomical data derived therefrom are used to determine the leg mechanical axis 208 from one or a plurality of anatomical indicators of a patient's limb prior to determining constitutional or pre-operative information, such as HKA for said patient. In this regard, a proximal portion and a distal portion of a patient's limb are typically identified. The patient's leg mechanical axis 208 may then be determined by projecting and extending an imaginary line between the identified portions, i.e., from the centers of the hip, knee, and ankle. Further, it would be appreciated that additional anatomical indicators therebetween may be used in determining the leg mechanical axis 208 and other information about a patient's limb and the relationship between the overall axes and the intercalated segments thereof (e.g., the femoral and tibial mechanical axes). It will be understood that a femoral mechanical axis typically refers to a line that extends from a center of a femoral head to a center of an intercondylar notch of a distal femur as assessed in a frontal or coronal plane.

It will be appreciated that the femoral mechanical axis 210 and/or the femoral joint line can be determined by any method or anatomical indicator/s known in the art. In particular examples, however, the one or a plurality of first anatomical indicators are selected from the group consisting of a central portion of a femoral head, a central portion of a distal femoral shaft, an intramedullary canal insertion point, the deepest portion of a trochlear groove and a central portion of an intercondylar notch. Further to this, the one or plurality of second anatomical indicators are suitably selected from the group consisting of a distal portion of a medial condyle and a distal portion of a lateral condyle.

As used herein, a tibial mechanical axis 212 generally refers to a line from a center of a tibial plateau to a center of a talus, although it will be understood that the tibial mechanical axis 212 and/or the tibial joint line can be determined by any method or anatomical indicator/s as are known in the art. In some examples, the one or a plurality of third anatomical indicators are selected from the group consisting of a central portion of a line extending between medial and lateral tibial spines, a central portion of a talus, a central portion of a proximal tibial shaft, and an anterior cruciate ligament tibial attachment point. By way of example, the central portion of the line extending between the medial and lateral tibial spines or intercondylar eminence can be determined, at least in part, by a line extending between a lateral intercondylar tubercle and a medial intercondylar tubercle. Accordingly, the central portion of the medial and lateral tibial spines may be the midpoint of this line extending between the two intercondylar tubercles.

Additionally, the one or plurality of fourth anatomical indicators is suitably selected from the group consisting of a proximal portion of a medial tibial plateau, a proximal portion of a lateral tibial plateau, a central portion of a lateral meniscus, and a central portion of a medial meniscus.

With regards to the femoral and/or tibial shafts, any central portion/s or point/s along the shaft, such as proximal, middle, or distal central portions, may be used as an anatomical indicator (i.e., first and/or third anatomical indicators) by the skilled person in performing the method of the present feature.

It will be readily understood that the present disclosure should not be limited to the specific examples of anatomical indicators provided herein (i.e., first, second, third, and/or fourth anatomical indicators). Rather, the term “anatomical indicator,” as used herein, refers to a readily identifiable feature, specific area, and/or landmark within or on a limb, such as an arm or leg, and/or a bone, such as a femur or tibia. In this regard, the anatomical indicators used for determining, for example, a leg mechanical axis 208, a tibial mechanical axis 212, a femoral mechanical axis 210, a femoral joint line, and a tibial joint line, are not to be limited to those anatomical indicators provided herein, but may also include other anatomical indicators as are known in the art.

At block 706, the target alignment of the patient's knee is determined. In some examples, a pre-surgical “hip-knee-ankle angle” (HKA) is computed, which refers to an angle formed by the femoral mechanical axis 210 and the tibial mechanical axis 212. A HKA is preferably measured or determined from a full-length lower-limb radiograph, and in healthy adults with a neutral alignment, the HKA is typically between 1.0 degrees and 1.5 degrees of varus (e.g., 1.0, 1.1, 1.2, 1.3, 1.4, 1.5 degrees and any range therein).

In some examples, the method further includes classifying the pre-operative alignment of the knee based at least in part on the HKA. For example, the alignment is classified as (a) valgus if the pre-operative HKA is positive in value; (b) varus if the pre-operative HKA is negative in value; or (c) neutral if the pre-operative HKA is substantially zero.

As will be appreciated by the skilled artisan, a varus alignment (e.g., a varus pre-operative alignment) refers to an inward angulation of a distal portion of a tibia on a coronal limb image, as measured by the HKA, whilst a valgus alignment (e.g., a valgus pre-operative alignment) refers to an outward angulation of a distal portion of a tibia on a coronal limb image, as measured by the HKA.

In some examples, a target alignment is determined. The target alignment may be the pre-operative alignment. The target alignment may be an adjusted pre-operative alignment (for example, degrees varus/valgus+/−an offset angle). In other examples, the target alignment may be determined based on various optimization techniques, for instance, based on kinematic simulation of the post-surgical knee of the patient.

In some examples, the method further includes determining one or more resection planes from at least partly the pre-operative HKA and/or the leg mechanical axis 208 at block 708. The resection planes may include an anterior resection plane, a posterior resection plane, and a distal resection plane of the femur and/or a proximal resection plane of the tibia.

Referring to the above, once the pre-operative alignment of the knee in question has been determined, any coronal realignment (if required) of the extended or flexed tibia relative to the tibial and/or femoral mechanical axes can then be performed on, for example, a patient-specific model of the patient's limb. Preferably, the degree and type of any deformity and/or defect, such as varus and valgus, of the knee joint in question are assessed on the patient-specific model. This then allows for a determination of the amount of varus or valgus rotation (i.e., medial or lateral rotation of the tibia relative to its corresponding femur in a coronal arc) that is required for angular correction of the tibia, femur, and/or the knee joint line relative to, for example, the tibial and/or femoral mechanical axes, so as to return the knee joint to the pre-operative alignment.

In some examples, the femoral resection plane 312 is determined as a plane that is perpendicular to the planned resultant leg mechanical axis 208. In various examples, the resulting femoral joint line relative to the femoral mechanical axis 210 may be the native varus-valgus or within an offset of the native varus-valgus. Alternatively, the femoral resection plane 312 is determined as a plane that is perpendicular to the planned resultant leg mechanical axis 208. In various examples, the resulting femoral joint line relative to the leg mechanical axis 208 may be the native varus-valgus or within an offset of the native degrees varus-valgus. In other examples, the femoral resection plane is determined as a plane that is perpendicular to the tibial mechanical axis (for an asymmetric tibial implant) or a femoral mechanical axis (for an asymmetric femoral implant).

In some examples, the tibial resection plane 314 is determined as a plane that is perpendicular to the planned resultant leg mechanical axis 208, In various examples, the resulting tibial joint line may be varus-valgus or within 1 or 2 degrees varus-valgus relative to the tibial mechanical axis 212. Alternatively, the tibial resection plane 314 is determined as a plane that is perpendicular to the planned resultant leg mechanical axis 208. In various examples, the resulting tibia joint line relative to the leg mechanical axis 208 may be the native varus-valgus or within an offset of native varus-valgus.

In some examples, the offset value may be about 1 degree, about 2 degrees, about 3 degrees, about 5 degrees, about 10 degrees, about 1 degree to about 2 degrees, or any value or range between any two of these values or ranges (including endpoints).

The resection planes 312 and 314 result in asymmetric resections 206 in the femur 202 and the tibia 204, as described herein.

At block 710, the surgical plan with the determined resection planes 312 and 314 is provided. The surgical plan can be provided by displaying the surgical plan on the CASS 100 via a graphical user interface (GUI), printing the surgical plan, or in any other manner including manual instruments that reference the hip and ankle centers or intramedullary canals. The surgeon and/or other users may review and/or edit the surgical plan in some examples. In some examples, the edits may be made to one or more input parameters, such as the patient anatomical data, which results in the CASS 100 computing updated resection planes 312, 314.

In some examples, the GUI displays the resection plane(s) overlaid on a digital image of the patient's anatomy of the limb. The surgeon can use the overlay as a guide to perform the resections.

In some examples, at block 712, the surgical plan is provided to a surgical computer 148 that performs the resections of the bones and/or the implant according to the computing resection planes 312, 314. The surgical computer 148 performs resections using robotic surgery in an autonomous manner.

A method for planning a knee arthroplasty procedure for a patient comprises obtaining patient-specific anatomical data of a limb, a prosthesis to be implanted in a knee of the limb; determining a leg mechanical axis of the limb; determining a target angle between a tibial mechanical axis and a tibial joint line (based, in some examples, on a pre-operative patient angle); determining a resection plane of a tibia bone to implant the prosthesis, the resection plane is perpendicular to the leg mechanical axis of the limb, and a resulting angle between a resulting tibial mechanical axis and the tibial joint line is within a predetermined threshold of the pre-operative angle; and providing a surgical plan depicting the resection plane to be used.

A method for guiding a surgeon performing a TKA comprises providing a surgical plan as described herein and providing a GUI in which the resection plane that is determined is overlaid on a digital image of the patient's anatomy of the limb.

A method for a robotic TKA includes determining a resection plane as described herein and inputting the resection plane into a surgical computer 148. The surgical computer 148 resects the bones (and/or the implant) according to the computed resection plane.

While various features and examples have been disclosed herein, other features and examples will be apparent to those skilled in the art. The various features and examples disclosed herein are for purposes of illustration and are not intended to be limiting, with the true scope and spirit being indicated by the following claims.

An executable application, as used herein, comprises code or machine-readable instructions for conditioning the processor to implement predetermined functions, such as those of an operating system, a context data acquisition system, or other information processing system, for example, in response to user command or input. An executable procedure is a segment of code or machine-readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

A graphical user interface (GUI), as used herein, comprises one or more display images generated by a display processor and enabling user interaction with a processor or other device and associated data acquisition and processing functions. The GUI also includes an executable procedure or executable application. The executable procedure or executable application conditions the display processor to generate signals representing the GUI display images. These signals are supplied to a display device that displays the image for viewing by the user. The processor, under the control of an executable procedure or executable application, manipulates the GUI display images in response to signals received from the input devices. In this way, the user may interact with the display image using the input devices, enabling user interaction with the processor or other devices.

The functions and process steps herein may be performed automatically or wholly or partially in response to user command. An activity (including a step) performed automatically is performed in response to one or more executable instructions or device operations without the user's direct initiation of the activity.

The system and processes of the figures are not exclusive. Other systems, processes, and menus may be derived in accordance with the principles of the present disclosure to accomplish the same objectives. Although the present disclosure has been described with reference to particular examples, it is to be understood that the examples and variations shown and described herein are for illustration purposes only. Modifications to the current design may be implemented by those skilled in the art without departing from the scope of the present disclosure.

Moreover, the following claims are hereby incorporated into this Detailed Description by this reference, with each claim standing on its own as a separate example of the present disclosure.

As used herein, an element or step recited in the singular and proceeded with the word “a” or “an” should be understood as not excluding plural elements or steps unless such exclusion is explicitly recited. Furthermore, references to “one example” of the present disclosure are not intended to be interpreted as excluding the existence of additional examples that also incorporate the recited features.

The phrases “at least one,” “one or more,” and “and/or,” as used herein, are open-ended expressions that are both conjunctive and disjunctive in operation. The terms “a” (or “an”), “one or more,” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure and do not create limitations, particularly as to the position, orientation, or use of this disclosure. Connection references (e.g., engaged, attached, coupled, connected, and joined) are to be construed broadly and may include intermediate members between a collection of elements relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only, and the dimensions, positions, order, and relative to sizes reflected in the drawings attached hereto may vary.

What is claimed is:

1. A total knee arthroplasty method, comprising:
determining a target joint angle for a knee joint of a patient;
determining a mechanical axis associated with the knee joint;
resecting at least one knee joint surface perpendicular to the mechanical axis, the at least one knee joint surface comprising at least one surface of one of a proximal tibia or a distal femur; and
implanting at least one asymmetric implant on the at least one knee joint surface, the at least one asymmetric implant comprising a base portion configured to be arranged on the at least one knee joint surface and an asymmetric articulating surface configured to implement the target joint angle for the post-surgical knee joint.

2. The method of claim 1, wherein the target joint angle is a pre-operative hip-knee-ankle (HKA) angle of the patient.

3. The method of claim 1, wherein the target joint angle is one of a pre-operative varus or valgus knee joint angle.

4. The method of claim 1, wherein the target joint angle is one of a pre-operative varus or valgus knee joint angle+/−an offset value.

5. The method of claim 1, wherein the mechanical axis comprises a leg mechanical axis.

6. The method of claim 1, wherein the at least one asymmetric implant is an asymmetric tibial implant, and the mechanical axis is a leg mechanical axis.

7. The method of claim 1, wherein the at least one asymmetric implant is an asymmetric femoral implant, and the mechanical axis is a leg mechanical axis.

8. The method of claim 1, wherein at least one asymmetric implant comprises an asymmetric tibial implant and an asymmetric femoral implant.

9. An asymmetric implant component, comprising:
a base portion configured to be arranged on a resected knee joint surface resected perpendicular to a mechanical axis associated with a knee joint of a patient; and
an asymmetric articulating surface configured to engage a corresponding opposite implant component and to implement a target joint angle for the post-surgical knee joint.

10. The asymmetric implant of claim 9, wherein the target joint angle is a pre-operative hip-knee-ankle (HKA) angle of the patient.

11. The asymmetric implant of claim 9, wherein the target joint angle is one of a pre-operative varus or valgus knee joint angle.

12. The asymmetric implant of claim 9, wherein the target joint angle is one of a pre-operative varus or valgus knee joint angle+/−an offset value.

13. The asymmetric implant of claim 9, wherein the mechanical axis comprises a leg mechanical axis.

14. The asymmetric implant of claim 9, wherein the at least one asymmetric implant is an asymmetric tibial implant, and the mechanical axis is a leg mechanical axis.

15. The asymmetric implant of claim 9, wherein the at least one asymmetric implant is an asymmetric femoral implant, and the mechanical axis is a leg mechanical axis.

16. The asymmetric implant of claim 9, wherein at least one asymmetric implant comprises an asymmetric tibial implant and an asymmetric femoral implant.

17. A computer-assisted surgical system, comprising:
a display device;
processing circuitry; and
a memory coupled to the processing circuitry, the memory comprising instructions that, when executed by the processing circuitry, cause the processing circuitry to:
receive a target joint angle for a knee joint of a patient,
receive a mechanical axis associated with the knee joint, and
determine a resection surface perpendicular to the mechanical axis, the resection surface comprising at least one surface of one of a proximal tibia or a distal femur,
determine an asymmetric implant for implanting on the resected surface, the asymmetric implant comprising a base portion configured to be arranged on the at least one knee joint surface and an asymmetric articulating surface configured to implement the target joint angle for the post-surgical knee joint, and
generate a surgical plan for implanting the asymmetric implant on the patient to achieve the joint angle.

18. The computer-assisted surgical system of claim 17, wherein the target joint angle is a pre-operative hip-knee-ankle (HKA) angle of the patient.

19. The computer-assisted surgical system of claim 17, wherein the target joint angle is one of a pre-operative varus or valgus knee joint angle.

20. The computer-assisted surgical system of claim 17, wherein the mechanical axis comprises a leg mechanical axis.

21. The computer-assisted surgical system of claim 17, wherein the at least one asymmetric implant is an asymmetric tibial implant, and the mechanical axis is a leg mechanical axis.

22. The computer-assisted surgical system of claim 17, wherein the at least one asymmetric implant is an asymmetric femoral implant, and the mechanical axis is a leg mechanical axis.

23. The computer-assisted surgical system of claim 17, wherein at least one asymmetric implant comprises an asymmetric tibial implant and an asymmetric femoral implant.
